(19) United States
(12) Patent Application Publication
(10) Pub. No.: US 2021/0290639 A1
(43) Pub. Date: Sep. 23, 2021

(54) IMMEDIATE RELEASE FORMULATION OF A TRIPLE COMBINATION OF ACTIVE PHARMACEUTICAL INGREDIENTS USEFUL IN THE TREATMENT OF POLYCYSTIC OVARY SYNDROME

(71) Applicants: HOSPITAL SANT JOAN DE DEU, Barcelona (ES); KATHOLIEKE UNIVERSITEIT LEUVEN, Leuven (BE)

(72) Inventors: Luis SOLER RANZANI, TERRASSA (ES); Laura MESTRE CERVANTES, TERRASSA (ES); Rosa TORRES FERRAZ, TERRASSA (ES)

(21) Appl. No.: 17/265,198

(22) PCT Filed: Aug. 1, 2019
(86) PCT No.: PCT/EP2019/070751

§ 371 Date: Feb. 1, 2021

(30) Foreign Application Priority Data
Aug. 2, 2018 (EP) 18382586.8

(51) Int. Cl. A61K 31/585; A61K 9/00; A61K 31/4439; A61K 31/155; A61K 9/28; A61K 9/20

(52) U.S. Cl. CPC A61K 31/585; A61K 9/0053; A61K 31/4439; A61K 9/2893; A61K 9/284; A61K 9/2031; A61K 9/2095; A61K 31/155

(57) ABSTRACT

Immediate release formulation of a triple combination of active pharmaceutical ingredients useful in the treatment of polycystic ovarysyndrome It relates to an immediate release formulation for oral administration, comprising; a) a combination of three active pharmaceutical ingredients which are spironolactone; pioglitazone or a salt thereof; and metformin or a salt thereof; and b) solid polyethylene glycol having an average molecular weight from 3350 to 8000 g/mol; wherein: each of the active pharmaceutical ingredients are present in a therapeutically effective amount; and the polyethylene glycol is present in an amount such that decreases the dissolution profiles in vitro of each one of the active pharmaceutical ingredients without modifying the disintegration time of the formulation compared with the dissolution profiles of each one of the active pharmaceutical ingredients of a formulation having the same composition but without the solid polyethylene glycol, as well as, to its preparation process.

This application claims the benefit of European Patent Application EP18382586 filed 2 August 2018.

The present invention relates to a composition which is useful in the treatment of prevention of polycystic ovary syndrome (PCOS) or PCOS-like conditions, more particularly, to an immediate release formulation of three active ingredients (APIs) which are spironolactone, pioglitazone and metformin containing an excipient able to modify the release profile in vitro of either active API acting alone without modifying the disintegration time of the formulation.

### BACKGROUND ART

Polycystic ovary syndrome (PCOS) is the most common endocrine disorder of women of reproductive age and has both hormonal and metabolic component. It is one of the leading causes of poor fertility. Signs and symptoms of PCOS include ovulatory dysfunction with irregular or no menstrual periods, heavy periods, hirsutism, acne, pelvic pain, and difficulty getting pregnant, Associated conditions include type 2 diabetes, obesity, obstructive sleep apnea, heart disease, mood disorders, and endometrial cancer. PCOS has a combination of both genetic and environmental factors. Risk factors include obesity, not enough physical exercise, and a family history of someone with the condition. Several professional groups have proposed different diagnostic criteria, for NIH criteria (National Institutes of Health) diagnosis is based on two of the following two findings: oligo-ovulation, high androgen levels. Other diagnostic criteria, such as Rotterdam, include the presence of ovarian cysts as a criteria for diagnosis. Cysts may be detectable by ultrasound. Other conditions that produce similar symptoms include adrenal hyperplasia, hypothyroidism, and hyperprolactinemia and are excluded from diagnosis.

Treatment may involve lifestyle changes such as weight loss and exercise. Birth control pills may help with improving the regularity of periods, excess hair growth, and acne. Metformin and anti-androgens may also help. Other typical acne treatments and hair removal techniques may be used. Efforts to improve fertility include weight loss, clomiphene, or metformin. In vitro fertilization is used by some in whom other measures are not effective.

There is currently no product approved worldwide for this indication. Many completed clinical studies and recommendations for treatment from endocrinology societies have shown the potential for one or even two of the proposed monocomponents to have some benefits in treating the most common signs and symptoms of PCOS. However, these treatments are often incomplete and do not address the multisystem derangements in these patients. Moreover, as there is an important and well described interplay and interaction between the hyperandrogenic and metabolic components of the disorder then a treatment targeting one sign or symptom is unlikely to address other disease aspects or reduce the risk of long term mobility and risks for additional complications.

WO2017/072243A1 discloses a fixed-dose combination (SPIOMET) of metformin hydrochloride (referred to as metformin or Met), spironolactone (Spi) and pioglitazone hydrochloride (referred to as pioglitazone or Pio) for use in the treatment of PCOS and PCOS-like conditions, including the treatment or prevention of sub fertility due to a low ovulation rate associated with liver steatosis in adolescent girls or women of childbearing age. It has been designed to tackle the main aspects that cause PCOS including the hyperandrogenic and metabolic factors of the condition. The SPIOMET treatment decreased the visceral and liver fat content to a greater extent than reported in previous studies investigating the effects of alternative insulin sensitizing treatments and the positive effect persisted after termination of the treatment. Available clinical data that supports the use of the three components in combination in PCOS or PCOS-like conditions has been obtained after the co-administration of the current individual commercial products off label (Aldactone®-Spi, Actos®-Pio, Dianben®-Met).

Both spironolactone and pioglitazone are BCS Class II (Biopharmaceutics Classification System) with low solubility and high permeability. Due to the low solubility of spironolactone and pioglitazone and the low content in the formulation of the 2 APIs (approximately 4.5% and 0.7%, respectively), in the document WO2017/072243A1, a wet granulation technology is suggested for formulating the triple combination together with: polyvinylpyrrolidone, sodium croscarmellose, microcrystalline cellulose, magnesium stearate, and polyvinyl alcohol.

Wet granulation is generally a preferred procedure in the manufacture of compressed tablets. However, when the present inventors tried to prepare a single unit composition of the three active pharmaceutical ingredients with the conventional excipients disclosed in the previous document, that is, polyvinyl pyirrolidone as a binder, sodium croscarmellose as a diluent, mycrocrystalline cellulose as a disgregant, and magnesium stearate as lubricant, found that the granulate show an important problem of compressibility and the tablets were highly friable, having low hardness even working with a high force of compression, and, besides, the tablets suffer from stripping. This technical difficulty in the compression may be due to the low compressibility of metformin hydrochloride, the disparity in the doses of the three active ingredients, and the high dose of metformin hydrochloride in the single unit form which also result in a quite voluminous units.

Therefore, It is considered desirable for those skilled in the art to provide a formulation of the three active ingredients formulated as immediate release formulation for oral administration to be able to bridge the existing evidence on the efficacy of the combination for PCOS treatment. It is also considered desirable to develop a non-process dependent formulation to be able to have a robust and scalable formulation.

### SUMMARY OF THE INVENTION

In the development of an immediate release formulation of three active ingredients (API) (spironolactone, pioglitazone and metformin), the present inventors found that by the addition of solid polyethylene glycol (PEG) having a molecular weight between 3350-8000 g/mol before the compression step, an optimal flowability and compaction properties of the blend are obtained, providing a non-process dependent formulation with a marked robustness and industrial scalability Thus, by addition of such PEG to a formulation comprising the triple combination of active ingredients together with other conventional excipients, the compressibility of the granule is notably increased using a lower compression force, i.e. a wider range of compression. Conversely, when the compression was carried out without the solid PEG, there were considerable difficulties to compress the formulation at industrial scale since it does not compress well in the required format. The immediate release formulation is prepared in a format appropriate to make it easily to swallow and divisible.

Furthermore, the use of solid polyethylene glycol (PEG) in the oral immediate release pharmaceutical formulation of the invention significantly decrease dissolution profiles in vitro compared with the dissolution profile of either active pharmaceutical ingredient (around 5-10%) without modifying physical attributes of the formulation such as the disintegration time when it is compared to the same formulation without PEG (see FIGS. 1-3 and Examples 3-6). The delay in the dissolution profile by the use of polyethylene glycol (PEG) in the formulation having the triple combination of APIs is surprising in view of the fact that usually polyethylene glycol acts as a water-soluble binder, and indeed it's known to increase release profile because of its structure. According to the Handbook of Pharmaceutical Excipients, Third Edition, Edited by Arthur H. Kibbe. Ph.D. (page 393), formulations having concentrations of PEG higher than 5% PEG may have an increase in the disgregation time. Also A. Anisha et al in Expert Opinion on drug delivery, vol. 13, no. 9, pp.1257-1275 discloses that PEGs can enhance the aqueous solubility or dissolution characteristics of poorly soluble compounds by conjugation or admixture with an appropriate PEG. It has been shown that solid dispersions of BCS Class 2 compounds with low molecular weight PEGs up to 6000 have markedly improved their dissolution rate (increase the % of the released drug). One of ordinary skill would not logically seek to add solid polyethylene glycol to the immediate release formulation of the three active ingredients since it would be expected an increase in the release profile. In contrast, it is found that the addition of solid PEG increases the hardness of the tablet without modifying the disintegration time. The increase in the hardness means that the tablet does not break, which is advantageous for the peculiar shape of the tablet to resist the coating step and even it could provide a reduced size formulation.

Therefore, the use of solid PEG allows preparing for the first time an immediate release pharmaceutical formulations of the three APIs in a single formulation per administration preferably once a day, which will enhance the adherence of patients to the treatment for PCOs, i.e. avoids taking three separated formulations, which is specially relevant for relatively young patient population not used to polymedication.

Accordingly, a first aspect of the present invention relates to an immediate release formulation for oral administration, comprising: a) a combination of three active pharmaceutical ingredients which are spironolactone or a salt thereof; pioglitazone or a salt thereof; and metformin or a salt thereof; and b) a solid polyethylene glycol having an average molecular weight from 3350 to 8000 g/mol; wherein: each of the active pharmaceutical ingredients are present in a therapeutically effective amount; and the polyethylene glycol is present in an amount such that decreases the dissolution profiles in vitro of each one of the active pharmaceutical ingredients without modifying the disintegration time of the formulation compared with the dissolution profiles of each one of the active pharmaceutical ingredients of a formulation having the same composition but without the solid polyethylene glycol).

A second aspect of the present invention relates to a process for preparing the immediate release formulation as defined above comprising: a) wet granulating the appropriate amounts of spironolactone, pioglitazone or a salt thereof, and metformin or a salt thereof with the appropriate amount of solid polyethylene glycol having an average molecular weight from 3350 to 8000 g/mol, and with one or more additional excipients, b) compressing the blend obtained in step a); c) optionally coating the formulation with a film-forming coating.

### BRIEF DESCRIPTION OF THE DRAWINGS

FIG. 1 shows comparative in vitro dissolution profiles of Table 1 and Table 2 SPIOMET formulations of batch size of 8.5 kg (G201801911 (with solid PEG) versus 6201801910 (without solid PEG) for spironolactone.

FIG. 2 shows comparative in vitro dissolution profiles of Table 1 and Table 2 SPIOMET formulations of batch size of 8.5 kg (G201801911 versus 6201801910) for metformin. FIG. 3 shows comparative in vitro dissolution profiles of Table 1 and Table 2 SPIOMET formulations of batch size of 8.5 kg (G201801911 versus G201801910) for pioglitazone.

### DETAILED DESCRIPTION OF PARTICULAR EMBODIMENTS

Spironolactone refers to a synthetic steroidal compound named 7α-Acetylthio-3-oxo-17α-pregn-4-ene-21,17β-carbolactone, which has the CAS number 52-01-7 and the formula (I) below. In most countries spironolactone is marketed within a drug formulation branded as Aldactone®.

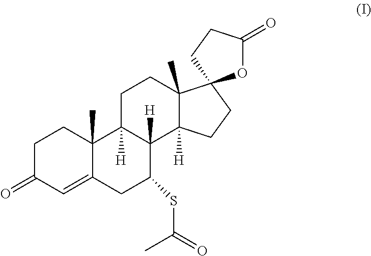

Pioglitazone refers to a compound of the class thiazolidinedione named ((±)-5-[p-[2-(5-Ethyl-2-pyridyl)ethoxy]benzyl]-2,4-thiazolidinedione, marketed as monohydrochloride and, which has the CAS number 112529-15-4 and formula (II) below. In most countries pioglitazone is marketed within a drug formulation branded as Actos®.

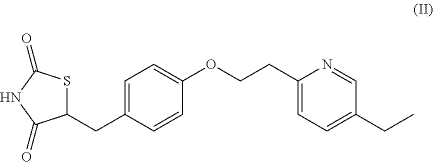 [SMILES: CCc1ccc(CCOc2ccc(CC3SC(=O)NC3=O)cc2)nc1]

Metformin refers to a compound named 1,1-Dimethylbiguanide marketed s monohydrochloride, which has the CAS number 1115-70-4 and the formula (III) below. In most countries metformin is marketed within a drug formulation branded as Dianben® or Glucophage®.

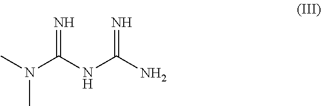

Other pharmaceutically acceptable salts of the previous compounds may be used for the purposes of the invention. As used herein, and unless otherwise specified, the term “pharmaceutically acceptable salt” refers to salts prepared from pharmaceutically acceptable non-toxic acids, including inorganic and organic acids. Examples of suitable non-toxic acids include hydrochloride, hydrochloric, hydrobromic, phosphoric, sulfuric, acetic, benzenosulfonic, benzoic, camphorsulfonic, citric, ethenesulfonic, formic, fumaric, lactic, maleic, malic, methanesulfonic, nicotinic, stearic, succinic, tartaric, p-toluensulfonic, and the like.

The compounds of formula (I), (II), and (III) may be in crystalline form either as free solvation compounds or as solvates (e.g. hydrates), or may be in the form of cocrystals and it is intended that these forms are within the scope of the present invention.

The term “solvate” refers to a molecular complex comprising the compounds mentioned above or a salt thereof, and a stoichiometric or non-stoichiometric amount of one or more solvent molecules bound by non-covalent intermolecular forces. When the one or more solvent molecules forming part of the molecular complex is water, the solvate is a hydrate.

The term “cocrystal” refers herein to a crystalline entity with at least two different components constituting the unit cell at room temperature (20-25 ° C.) and interacting by weak interactions. Thus, in a cocrystal the active pharmaceutical ingredient crystallizes with one or more neutral components. The cocrystal may include one or more solvent molecules in the crystal lattice. The term “weak interaction” refers herein as an interaction which is neither ionic nor covalent, and includes for example: hydrogen bonds, van der Waals interactions, and rr-rr stacking.

Polyethylene glycol (PEG), also known as macrogol, is a polyether composed of repeated ethylene glycol units —[(CH2—CH2—O)n]—. CAS n°: 25322-68-3 (all PEGs). Ph. Eur n°: 1444E. Commercial PEGs are available with different degrees of polymerization and activated functional groups. For the purposes of the invention it is used solid PEG having an average molecular weight comprised between 3350 and 8000 g/mol (i.e. PEG3350-PEG 8000), preferably PEG having an average molecular weight of 4000 g/mol (PEG 4000). This polyethylene glycol is also named Polyglykol 4000 PS (powder spray) or Polyethylene glycol 4000 or Macrogol 4000. The average molecular weight of the polyethylene glycol may be determined by gel permeation chromatography using the universal calibration method (cf. European pharmacopeia 9.0, 2.2.30).

The term “percentage (%) by weight” refers to the percentage of each ingredient of the formulation in relation to the total weight of the formulation. If the formulation is a coated formulation, then the total weight refers to the total weight of the coated formulation. If the formulation is an uncoated formulation, then the total weight refers to the total weight of the uncoated formulation.

The expression “therapeutically effective amount” as used herein, refers to the amount of a compound that, when administered, alone or in combination with other active compounds, is sufficient to prevent development of, or alleviate to some extent, one or more of the symptoms of the disease which is addressed. The particular dose of compound administered according to this invention will of course be determined by the particular circumstances surrounding the case, including the compound administered, the route of administration, the particular condition being treated, and the similar considerations.

The expression “pharmaceutically acceptable excipients or carriers” refers to pharmaceutically acceptable materials, compositions or vehicles. Each component must be pharmaceutically acceptable in the sense of being compatible with the other ingredients of the immediate release formulation. It must also be suitable for use in contact with the tissue or organ of humans and animals without excessive toxicity, irritation, allergic response, immunogenicity or other problems or complications commensurate with a reasonable benefit/risk ratio.

As mentioned above, it is provided an oral immediate release formulation for oral administration, comprising; a) a combination of three active pharmaceutical ingredients which are spironolactone; pioglitazone or a salt thereof; and metformin or a salt thereof; and b) a solid polyethylene glycol having an average molecular weight from 3350 to 8000 g/mol; wherein: each of the active pharmaceutical ingredients are present in a therapeutically effective amount; and the polyethylene glycol is in an amount such that decrease the dissolution profiles in vitro of each one of the active pharmaceutical ingredients without modifying the disintegration time of the formulation compared with the dissolution profiles of each one of the active pharmaceutical ingredients of a formulation having the same composition but without the solid polyethylene glycol. The oral immediate release formulation of the present invention is an oral disintegrating tablet.

In a particular embodiment, optionally in combination with one or more features of the particular embodiments defined above or below, the oral immediate release formulation of the present invention is that which comprises an outer coating: In another particular embodiment, optionally in combination with one or more features of the particular embodiments defined above or below, the oral immediate release formulation of the present invention is that where the outer coating is a film-forming coating.

In another particular embodiment, optionally in combination with one or more features of the particular embodiments defined above or below, the oral immediate release formulation of the present invention is that wherein the formulation is a tablet or capsule, preferably a tablet.

In another particular embodiment, optionally in combination with one or more features of the particular embodiments defined above or below, the oral immediate release formulation is that which is a film coated tablet.

In a particular embodiment, it is provided an immediate release formulation for oral administration, comprising a) a combination of three active pharmaceutical ingredients which are spironolactone; pioglitazone or a salt thereof; and metformin or a salt thereof; and b) a solid polyethylene glycol having an average molecular weight from 3350 to 8000 g/mol; wherein: each of the active pharmaceutical ingredients are present in a therapeutically effective amount; and the polyethylene glycol is present in an amount from 4 to 10% by weight with respect to the total weight of the formulation. When the formulation is coated, then it refers to the total weight of the coated formulation.

In another particular embodiment, optionally in combination with one or more features of the particular embodiments defined above or below, the oral immediate release formulation of the present invention is that wherein the polyethylene glycol is present in an amount from 6 to 8% by weight with respect to the total weight of the coated formulation.

In another particular embodiment, optionally in combination with one or more features of the particular embodiments defined above or below, the oral immediate release formulation of the present invention is that wherein the polyethylene glycol is present in an amount of from 7% by weight with respect to the total weight of the coated formulation.

In another particular embodiment, optionally in combination with one or more features of the particular embodiments defined above or below, the oral immediate release formulation of the present invention is that where the solid polyethylene glycol (PEG) is polyethylene glycol having an average molecular weight of 4000-6000 g/mol. In another particular embodiment, optionally in combination with one or more features of the particular embodiments defined above or below, the oral immediate release formulation of the present invention is that where the solid polyethylene glycol (PEG) is polyethylene glycol having an average molecular weight of 4000 g/mol.

In a particular embodiment, optionally in combination with one or more features of the particular embodiments defined above or below, the oral immediate release formulation of the present invention is that which further comprises at least one additional pharmaceutical excipient selected from the group consisting of: a binder, a diluent, a disintegrant, and a lubricant.

In another particular embodiment, optionally in combination with one or more features of the particular embodiments defined above or below, the oral immediate release formulation of the present invention is that which comprises: at least a binder, at least a diluent, at least a disintegrant, and at least a lubricant.

Examples of binders appropriate for the present invention are polyvinylpyrrolidone, hydroxypropylmethylcellulose, hydroxypropylcellulose, or carboxymethylcellulose.

Examples of diluents appropriate for the present invention are microcrystalline cellulose, silicifed microcrystalline cellulose, powdered cellulose, anhydrous lactose, lactose monohydrate, modified lactose, dibasic calcium phosphate, tribasic calcium phosphate, maize starch, pregelatinized starch, calcium carbonate, sucrose, glucose, dextrates, dextrins, dextrose, fructose, lactitol, mannitol, sorbitol, or starch.

Examples of disintegrants appropriate for the present invention are croscarmellose sodium, crospovidone, sodium starch glycolate, corn starch, potato starch, calcium silicated, or low substituted hydroxypropyl cellulose.

Examples of lubricants appropriate for the present invention are magnesium stearate, magnesium lauryl sulfate, sodium stearyl fumarate, sucrose esters, fatty acids, or stearic acid.

Additionally, the compositions of the present invention may contain other ingredients, such as fragrances, colorants, and other components known in the state of the art for use in formulations for oral administration.

In another particular embodiment, optionally in combination with one or more features of the particular embodiments defined above or below, the oral immediate release formulation of the present invention is that which comprises a binder which is polyvinylpyrrolidone; a diluent which is microcrystalline cellulose, a disintegrant which is croscarmellose sodium, and lubricant which is magnesium stearate.

In another particular embodiment, the immediate release formulation of the present invention is that where the binder is in an amount from 5.5 -7.5% by weight, the diluent is in an amount from 1.5 to 3% by weight, the disgregant is in an amount from 3 to 4.5% by weight, and the lubricant is in an amount from 0.5 to 1.5% by weight, with respect to the total weight of the formulation. When the formulation is coated, then it refers to the total weight of the coated formulation.

In another particular embodiment, optionally in combination with one or more features of the particular embodiments defined above or below, the oral immediate release formulation of the present invention is that where: the spironolactone, the pioglitazone or a salt thereof, and the metformin or a salt thereof, are the only active pharmaceutical ingredients of the formulation.

In another particular embodiment, optionally in combination with one or more features of the particular embodiments defined above or below, the oral immediate release formulation of the present invention is that where: the active pharmaceutical ingredients are spironolactone, pioglitazone hydrochloride; and metformin hydrochloride.

In another particular embodiment, optionally in combination with one or more features of the particular embodiments defined above or below, the oral immediate release formulation of the present invention is that wherein: a) the therapeutically effective amount of spironolactone is 4-5% by weight; b) the therapeutically effective amount of piogllitazone or a salt thereof is 0.5-1% by weight; and c) the therapeutically effective amount of metformin or a salt thereof is 73-75% by weight, with respect to the total weight of the formulation. In another particular embodiment, the oral immediate release formulation of the present invention is that wherein: a) the therapeutically effective amount of spironolactone or a salt thereof is 4.3t% by weight; b) the therapeutically effective amount of piogllitazone or a salt thereof is 0.7% by weight; and c) the therapeutically effective amount of metformin or a salt thereof is 73% by weight, with respect to the total weight of the formulation (coated formulation).

In a particular embodiment, optionally in combination with one or more features of the particular embodiments defined above or below, the oral immediate release formulation of the present invention is that where: a) the therapeutically effective amount of spironolactone is from 25 to 100 mg; b) the therapeutically effective amount of pioglitazone or a salt thereof is 5-15 mg; and c) the therapeutically effective amount of metformin or a salt thereof is 500-1500 mg. In another particular embodiment, optionally in combination with one or more features of the particular embodiments defined above or below, the immediate release formulation of the present invention typically comprises between 30-60 mg of spironolactone, 5-10 mg of pioglitazone hydrochloride, 600-1000 mg metformin hydrochloride. Generally, the previous amounts correspond to a daily dose of each of the active ingredients.

It is preferred that a daily dose of the immediate release formulation according to the present invention comprising the required daily dose of spironolactone, pioglitazone or a salt thereof, and metformin or a salt thereof is formulated in a unit single dose, i.e. comprises a therapeutically effective amount of each active pharmaceutical ingredient for a single dose administration: Particularly, the unit single dose are single tablets or capsules, which can be coated and which can be readily ingested.

In another particular embodiment, the oral immediate release formulation of the present invention is that which is a film-coated tablet per administration once a day, wherein a) the spironolactone is in an amount of 50 mg; b) the pioglitazone hydrochloride is in an amount of 8.3 mg (8.3 mg Pioglitazone HCl corresponds to 7.5 mg of Pioglitazone base); c) the metformin hydrochloride is in an amount of 850 mg; and d) the polyethylene glycol 4000 is in amount of 80 mg. The dose of pioglitazone is around half of the commercial one (7.5 mg vs.15 mg), the dose of metformin is also lower (850 mg vs. the normal dose that is two tablets of 850mg per day or 1 tablet per day of 1000 mg), the dose of spironolactone is 50 mg vs. the commercial one (25 or 100 mg).

In another particular embodiment, the oral immediate release formulation of the present invention further comprises: e) 76.8 mg of polyvinylpyrrolidone; f) 26.4 mg of microcrystalline cellulose; g) 42.8 mg of croscarmellose sodium; and h) 15.7 mg of magnesium stearate.

The immediate release formulation of the present invention can be prepared according to methods well known in the state of the art. In a particular embodiment, the immediate release formulation of the present invention is prepared by wet granulation. The oral immediate release formulation of the present invention, wherein the formulation is prepared by wet granulation is also part of the invention.

In a particular embodiment, the immediate release formulation of the present invention is prepared by a process comprising the steps of: a) wet granulating the appropriate amounts of spironolactone, pioglitazone or a salt thereof, and metformin or a salt thereof with the appropriate amount of solid polyethylene glycol having an average molecular weight from 3350 to 8000 g/mol, and with one or more additional excipients; b) compressing the mixture; c) optionally coating the formulation with a film-forming coating. The term appropriate amounts of spironolactone, pioglitazone or a salt thereof, and metformin or a salt thereof refers to the therapeutically effective amounts of each of the active pharmaceutical ingredients. The appropriate amount of polyethylene glycol is such that decreases the dissolution profiles in vitro of each one of the active pharmaceutical ingredients without modifying the disintegration time of the formulation compared with the dissolution profiles of each one of the active pharmaceutical ingredients of a formulation having the same composition but without the solid polyethylene glycol.

In a particular embodiment, the immediate release formulation of the present invention can be prepared by a process comprising the steps of: a) wet granulating the active pharmaceutical ingredients with solid polyethylene glycol having an average molecular weight from 3350 to 8000 g/mol; wherein the polyethylene glycol is in an amount from 6 to 8% by weight with respect to the total weight of the formulation, and with one or more additional excipients; b) compressing the mixture; and c) optionally coating the formulation with a film-forming coating.

The use of solid PEG in the immediate release formulation of the present invention, increases the compressibility of the granule using a wide range of compression force at industrial scale, generally the compression range being from 18 to 32 KN. This wide range is advantageous since it means that any small variation on the equipment when scaling the process, does not affect the process, which ensures the robustness of the compression.

In a particular embodiment, a suspension of a pigmented film coating based on polymer with the following composition Polyvinyl alcohol (PVA) partially hydrolysed, Talc, Titanium dioxide, Polyethylene glycol, Red iron oxide previously dispersed in purified water is used to coat the formulation.

In another particular embodiment, the process includes mixing metformin hydrochloride, spironolactone, pioglitazone hydrochloride, povidone k-30 and half of the croscarmellose sodium; then granulating the mixture with purified water, sieving the wet granulate, drying the sieved wet granulate, sieving the dried granulate, mixing it with croscarmellose sodium, polyethylene glycol 4000 PS, microcrystalline cellulose, and magnesium stearate and tablet the final blend. Finally, film-coat the cores thus obtained with a suspension of the pigmented film coating based on PVA polymer previously dispersed in purified water.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples and drawings are provided by way of illustration, and they are not intended to be limiting of the present invention. Furthermore, the present invention covers all possible combinations of particular and preferred embodiments described herein.

### EXAMPLES

### Example 1

### Preparation of the SPIOMET Immediate Release Oral Formulation with Polyethylene Glycol 4000

The immediate release oral formulation of the present invention was prepared by wet granulation followed by compression and film coating following the method illustrated below:

1. Metformin hydrochloride is placed it in the high shear and mix 10 min.

2. Put together Spironolactone, Pioglitazone hydrochloride, Povidone k-30 and half of the croscarmellose sodium and place the mixture in the high shear mixer together with Metformin hydrochloride

3. Mix the blend of step 2 in the high shear mixer

4. Granulate the mixture of step 3 in the high shear mixer with purified water.

5. Sieve the wet granulate from step 4

6. Dry in a fluid bed the wet granulate sieved from step 5.

7. Sieve the dry granulate from step 6.

8. Mix the sieved dried granulate with the previously sieved croscarmellose sodium, Polyethylene glycol 4000 PS and microcrystalline cellulose.

9. Mix the blend of step 8 with the previously sieved magnesium stearate.

10. Tablet the final blend obtained in step 9.

11. Film-coat the cores obtained in step 10 with a suspension of pigmented film coating based on PVA polymer previously dispersed in purified water.

Table 1 below show the content of the immediate release formulations prepared according to the steps above at two different scales 8.5 kg and 40 kg:

TABLE 1

| List of components of SPIOMET immediate release | mg/film-coated | |
|---|---|---|
| oral formulation with PEG and its function | tablets | % in the film |
| Batch size 8.5 Kg | G201801911 | coated tablet |
| Batch size 40 Kg | H1756A | (w/w) |
| Metformin HCl (active substance) | 850.00 | 72.56% |
| Spironolactone (active substance) | 50.00 | 4.27% |
| Pioglitazone HCl (active substance) | 8.27(1) | 0.71% |
| Povidone K-30 (binder) | 76.80 | 6.56% |
| Microcrystalline cellulose (diluent) | 26.38 | 2.25% |
| Croscarmellose sodium (disintegrant) | 42.80 | 3.65% |
| Polyethylene glycol 4000 PS (binder, plasticizer) | 80.00 | 6.83% |
| Magnesium stearate (lubricant) | 15.75 | 1.34% |
| Purified water(2) (solvent) | q.s.(2) | — |
| Total core weight | 1150.00 mg | — |
| pigmented film coating based on PVA polymer | 21.40 | 1.83 |
| Purified water(2) (solvent) | q.s.(2) | — |
| Total weight | 1171.40 mg | 100% |

(1)8.27 mg Pioglitazone HCl corresponds to 7.5 mg of Pioglitazone base
(2)Evaporated during the process. Not present in the final formulation

### Example 2

### Preparation of the SPIOMET Immediate Release Oral Formulation without Polyethylene Glycol 4000

The immediate release oral formulation of the present invention was prepared by wet granulation followed by compression and film coating following the method illustrated below:

1. Metformin hydrochloride is placed it in the high shear and mix 10 min.

2. Put together Spironolactone, Pioglitazone hydrochloride, Povidone k-30 and half of the croscarmellose sodium and place the mixture in the high shear mixer together with Metformin hydrochloride

3. Mix the blend of step 2 in the high shear mixer

4. Granulate the mixture of step 3 in the high shear mixer with purified water.

5. Sieve the wet granulate from step 4

6. Dry in a fluid bed the wet granulate sieved from step 5.

7. Sieve the dry granulate from step 6.

8. Mix the sieved dried granulate with the previously sieved croscarmellose sodium and microcrystalline cellulose.

9. Mix the blend of step 8 with the previously sieved magnesium stearate.

10. Tablet the final blend obtained in step 9.

11. Film-coat the cores obtained in step 10 with a suspension of pigmented film coating based on PVA polymer previously dispersed in purified water.

Table 2 below show the content of the immediate release formulations without polyethylene glycol 4000 prepared according to the steps at two different scales 8.5 kg and 40 kg.

Note that the 40 kg scale-up batch the resulted granules and the final mixture couldn't be compressed into tablets because it turned out to be a process dependent formulation where granulation step is critical.

TABLE 2

| List of components of SPIOMET immediate release | mg/film-coated | |
|---|---|---|
| oral formulation without PEG and its function | tablets | % in the film |
| Batch size 8.5 Kg | G201801910 | coated tablet |
| Batch size 40 Kg | H1756B | (w/w) |
| Metformin HCl (active substance) | 850.00 | 77.88% |
| Spironolactone (active substance) | 50.00 | 4.58% |
| Pioglitazone HCl (active substance) | 8.27(1) | 0.76% |
| Povidone K-30 (binder) | 76.80 | 7.04% |
| Microcrystalline cellulose (diluent) | 26.38 | 2.42% |
| Croscarmellose sodium (disintegrant) | 42.80 | 3.92% |
| Magnesium stearate (lubricant) | 15.75 | 1.44% |
| Purified water(2) (solvent) | q.s.(2) | — |
| Total core weight | 1070.00 mg | — |
| pigmented film coating based on PVA polymer | 21.40 | 1.96% |
| Purified water(2) (solvent) | q.s.(2) | |
| Total weight | 1091.40 mg | 100% |

(1)8.27 mg Pioglitazone HCl corresponds to 7.5 mg of Pioglitazone base
(2)Evaporated during the process. Not present in the final formulation

### Example 3

### Dissolution test of spironolactone of SPIOMET immediate release oral formulation

Dissolution test of spironolactone was performed according to EP 2.9.3 Dissolution test for solid dosage forms, current edition.

Dissolution medium: Buffer solution pH 4.5 (According to EP 5.9.17 Recommendations on methods for dosage forms testing, current edition) with 0.5% tween.

Apparatus 2 (EP 2.9.3): Paddle

Volume: 1000 mL

Temperature: 37±0.5° C.

Stirring speed: 75 rpm

Sampling times: 5, 10, 15, 30, 45 and 60 minutes.

The content of Spironolactone from the extracted samples is analyzed by reverse-phase

HPLC with UV detection method.

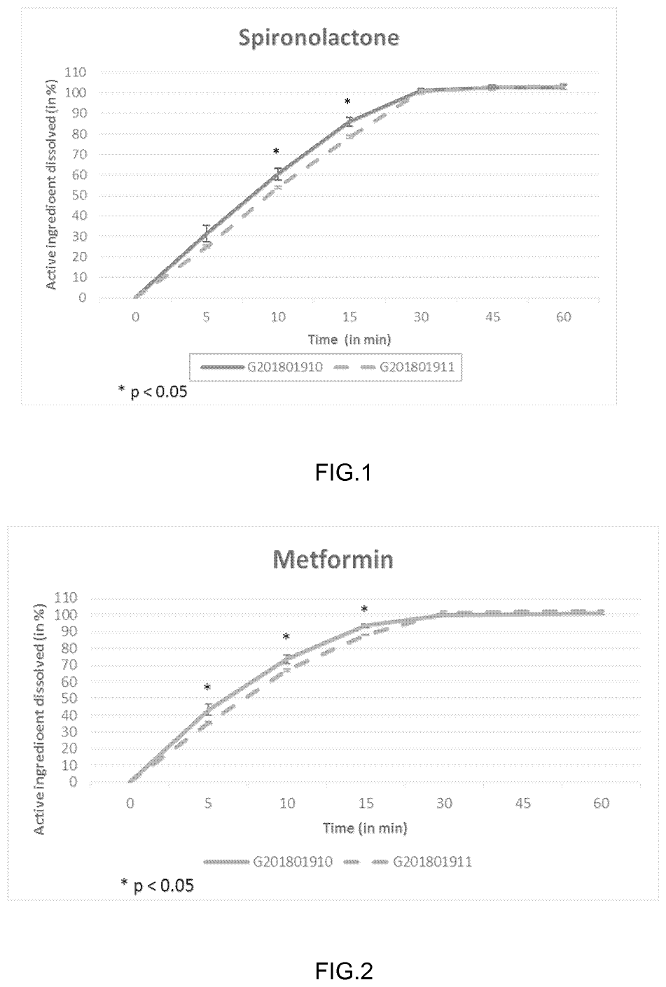

FIG. 1 shows comparative in vitro dissolution profiles of Table 1 and Table 2 SPIOMET formulations of batch size of 8.5 kg (G201801911 versus G201801910) for spironolactone. These results in FIG. 1 show that the release profile of spironolactone is significantly decreased (p<0.05) at 10 and 15 minutes in SPIOMET immediate release formulation with PEG (G201801911) compared with the same formulation without PEG (G201801910). The results are statistically significant.

### Example 4

### Dissolution test of Metformin of SPIOMET Immediate Release Oral Formulation

Dissolution test of metformin was performed according to EP 2.9.3 Dissolution test for solid dosage forms, current edition.

Dissolution medium: Buffer solution pH 4.5 (According to EP 5.9.17 Recommendations on methods for dosage forms testing, current edition) with 0.5% tween.

Apparatus 2 (EP 2.9.3): Paddle

Volume: 1000 mL

Temperature: 37±0.5° C.

Stirring speed: 75 rpm

Sampling times: 5, 10, 15, 30, 45 and 60 minutes.

The content of Metformin from the extracted samples is analyzed by reverse-phase HPLC with UV detection method.

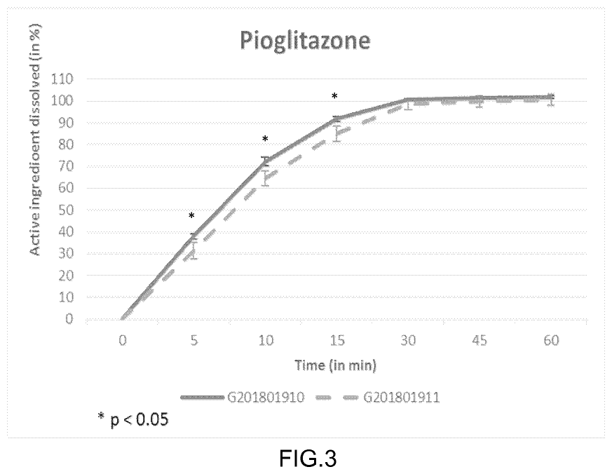

FIG. 2 shows comparative in vitro dissolution profiles of Table 1 and Table 2 SPIOMET formulations of batch size of 8.5 kg (G201801911 versus 201801910) for metformin. These results in FIG. 2 show that the release profile of metformin is significantly decreased (p<0.05) at 5, 10 and 15 minutes in SPIOMET immediate release formulation with PEG (G201801911) compared with the same formulation without PEG (G201801910). The results are statistically significant.

### Example 5

### Dissolution Test of Pioglitazone of SPIOMET Immediate Release Oral Formulation

The dissolution test of pioiglitazone was performed According to EP 2.9.3 Dissolution test for solid dosage forms, current edition.

Dissolution medium: Buffer solution pH 3.0 (see preparation of the solution).

Preparation of buffer solution pH3.0: Weight 8.5g of citric acid monohydrate and 3.2 g of NaOH pellets, dissolve with 800.0 mL of purified water, add 4.4 mL HCl 37% and shake 20 minutes. Dilute to 1000.0 mL with purified water. Adjust to pH3.0±0.05 with HCl 1 M.

Apparatus 2 (EP 2.9.3.): Paddle

Volume: 1000 mL

Temperature: 37±0.5° C.

Stirring speed: 100 rpm

Sampling times: 5, 10, 15, 30, 45 and 60 minutes.

The content of Pioglitazone from the extracted samples is analyzed by reverse-phase HPLC with UV detection method.

FIG. 3 show comparative in vitro dissolution profiles of Table 1 and Table 2 SPIOMET formulations of batch size of 8.5 kg (G201801911 versus G201801910) for pioglitazone.

These results in FIG. 3 show that the release profile of pioglitazone is significantly decreased (p<0.05) at 5, 10 and 15 minutes in SPIOMET immediate release formulation with PEG (G201801911) compared with the same formulation without PEG (G201801910). The results are statistically significant.

### Example 6

### Disintegration Test of SPIOMET Immediate Release Oral Formulation

The disintegration test was performed according to Ph. Eur. 2.9.1 current edition (Disintegration of Tablets and capsules).

Tables 3 and 4 show disintegration data obtained with the SPIOMET formulations with PEG (G201801911, H1756A) and without PEG (G201801910), respectively.

The results in Table 3 and Table 4 show that there's no difference in the physical parameter (disintegration) of SPIOMET immediate release formulation with PEG (G201801911) and formulation without PEG (G201801910), despite the difference found between both formulations on the release profile.

TABLE 3

| | |
|---|---|
| Film coated tablets | SPIOMET immediate release formulation |
| IPC-5 | with PEG |

| Batch number | G201801911 | H1756A |
|---|---|---|
| Batch size | 8.5 kg | 40 kg |
| Disintegration: | | |
| Mean (min) | <11 | <11 |
| N | 6 | 6 |

TABLE 4

| | |
|---|---|
| Film coated tablets | SPIOMET immediate release formulation |
| IPC-5 | without PEG |

| Batch number | G201801910 | H1756B(1) |
|---|---|---|
| Batch size | 8.5 kg | 40 kg |
| Disintegration: | | |
| Mean (min) | <11 | — |
| N | 6 | — |

(1)Note:
not enough hardness was achieved (<140N) to pass friability test. Hence, no tablets were obtained

### REFERENCES CITED IN THE APPLICATION

WO2017/072243A1

Handbook of Pharmaceutical Excipients, Third Edition, Edited by Arthur H. Kibbe. Ph.D. (page 393)

A. Anisha et al in Expert Opinion on drug delivery, vol. 13, no. 9, pp.1257-1275

European Pharmacopea 9.0, 2.2.30

1. An immediate release formulation for oral administration, comprising;
a) a combination of three active pharmaceutical ingredients which are spironolactone;
piogllitazone or a salt thereof; and metformin or a salt thereof; and
b) solid polyethylene glycol having an average molecular weight selected from 3350 to 8000 g/mol;
wherein:
each of the active pharmaceutical ingredients are present in a therapeutically effective amount; and
the polyethylene glycol is present in an amount that decreases the dissolution profiles in vitro of each one of the active pharmaceutical ingredients without modifying the disintegration time of the formulation when compared with the dissolution profiles of each one of the active pharmaceutical ingredients of a formulation having the same composition but without the solid polyethylene glycol.

2. The oral immediate release formulation according to claim 1, wherein the formulation comprises an outer coating which is a film-forming coating.

3. The oral immediate release formulation according to claim 1, wherein the formulation is a tablet.

4. The immediate release formulation according to claim 3, wherein the polyethylene glycol is in an amount selected from 4 to 10% by weight with respect to the total weight of the formulation.

5. The oral immediate release formulation according to claim 4, wherein the solid polyethylene glycol is present in an amount selected from 6 to 8% by weight with respect to the total weight of the formulation.

6. The oral immediate release formulation according to claim 5, wherein the solid polyethylene glycol (PEG) is polyethylene glycol having an average molecular weight selected from 4000-6000 g/mol.

7. The oral immediate release formulation according to claim 6, wherein the solid polyethylene glycol (PEG) is polyethylene glycol having an average molecular weight of 4000 g/mol.

8. The oral immediate release formulation according to claim 7, which further comprises at least one additional pharmaceutical excipient selected from the group consisting of: a binder, a diluent, a disintegrant.

9. The oral immediate release formulation according to claim 8, wherein it comprises
a binder which is polyvinylpirrolidone; a diluent which is microcrystalline cellulose, a disintegrant which is croscarmellose sodium, and lubricant which is magnesium stearate.

10. The oral immediate release formulation according to claim 9, wherein the active pharmaceutical ingredients are spironolactone, pioglitazone hydrochloride, and metformin hydrochloride.

11. The oral immediate release formulation according to claim 1, wherein:
a) the therapeutically effective amount of spironolactone is selected from 25 to 100 mg;
b) the therapeutically effective amount of pioglitazone or the salt thereof is selected from 5-15 mg; and
c) the therapeutically effective amount of metformin or the salt thereof is selected from 500-1500 mg.

12. The immediate release formulation according to claim 1, comprising the binder, the diluent, the disintegrant, and the lubricant, and wherein the binder is in an amount selected from 5.5 -7.5% by weight, the diluent is in an amount selected from 1.5 to 3% by weight, the disintegrant is in an amount selected from 3 to 4.5% by weight, and the lubricant is in an amount selected from 0.5 to 1.5% by weight, with respect to the total weight of the formulation.

13. The oral immediate release formulation according to claim 1, which is a film-coated tablet for per administration once a day, wherein
a) the spironolactone is in an amount of 50 mg;
b) the pioglitazone hydrochloride is in an amount of 8.3 mg;
c) the metformin hydrochloride is in an amount of 850 mg; and
d) the polyethylene glycol 4000 is in an amount of 80 mg.

14. The oral immediate release formulation according to claim 13, further comprising:
e) 76.8 mg of polyvinylpyrrolidone;
f) 26.4 mg of microcrystalline cellulose;
g) 42.8 mg of croscarmellose sodium; and
h) 15.7 mg of magnesium stearate.

15. A process for preparing the immediate release formulation according to claim 1, comprising:
a) wet granulating the appropriate amounts of spironolactone, pioglitazone or the salt thereof, and metformin or the salt thereof with the appropriate amount of solid polyethylene glycol having an average molecular weight selected from 3350 to 8000 g/mol, and with one or more additional excipients;
b) compressing the mixture; and
c) optionally coating the formulation with a film-forming coating.

16. The oral immediate release formulation according to claim 1, wherein the solid polyethylene glycol is present in an amount selected from 4 to 10% by weight with respect to the total weight of the formulation.

17. The oral immediate release formulation according to claim 2, wherein the formulation is a tablet.

18. The oral immediate release formulation according to claim 17, wherein the solid polyethylene glycol is present in an amount selected from 4 to 10% by weight with respect to the total weight of the formulation.

19. The oral immediate release formulation according to claim 18, wherein the solid polyethylene glycol (PEG) is polyethylene glycol having an average molecular weight selected from 4000-6000 g/mol.

20. The oral immediate release formulation according to claim 19, wherein the active pharmaceutical ingredients are spironolactone, pioglitazone hydrochloride, and metformin hydrochloride.
